(19) United States
(12) Patent Application Publication
(10) Pub. No.: US 2023/0181857 A1
(43) Pub. Date: Jun. 15, 2023

(54) Positive Airway Pressure Mask with Reduced Airflow During Exhalation

(71) Applicant: Encite LLC, Burlington, MA (US)

(72) Inventor: Stephen A. Marsh, Carlisle, MA (US)

(21) Appl. No.: 17/890,383

(22) Filed: Aug. 18, 2022

Related U.S. Application Data
(60) Provisional application No. 63/255,496, filed on Oct. 14, 2021.
(60) Provisional application No. 63/359,923, filed on Jul. 11, 2022.

(51) Int. Cl. A61M 16/06

(52) U.S. Cl. CPC A61M 16/0666; A61M 16/0683; A61M 2210/0618

(57) ABSTRACT

A positive air way pressure mask includes a mask body having an inlet and an outlet, a nasal interface supported on the body, the nasal interface having air passages, with the nasal interface coupling a user's nose to the outlet of the continuous positive air way pressure mask body, and an air switch device having an input port, an output port, and a diversion port, with the input port receiving air from a continuous positive airway pressure machine and delivering the received air to the output port during an inhalation, and that switches the air from the positive airway pressure machine from the inlet to the diversion port to expel diverted air to ambient during an exhalation. Also disclosed is an alternative voice coil actuated air switch device having an input port and an output port, with the input port receiving air from a continuous positive airway pressure machine and delivering the received air to the output port during an inhalation, and that inhibits the air from the positive airway pressure machine from entering the voice coil actuated air switch during an exhalation.

### CLAIM OF PRIORITY

This application claims priority under 35 USC § 119(e) to U.S. Provisional Patent Application Ser. No. 63/255,496, filed on Oct. 14, 2021, and entitled “Positive Airway Pressure Mask with Reduced Airflow During Exhalation” and to U.S. Provisional Patent Application Ser. No. 63/359,923, filed on Jul. 11, 2022, and entitled “Positive Airway Pressure Mask with Reduced Airflow During Exhalation,” the entire contents of which are hereby incorporated by reference.

### BACKGROUND

This specification relates to continuous positive airway pressure (CPAP) machines, and more particularly to CPAP masks used with CPAP machines.

Obstructive sleep apnea (OSA) is a disorder that is characterized by recurrent episodes of complete or partial obstruction of the upper airway leading to a reduction or an absence of breathing cycles during sleep. These episodes are termed “apneas.” As a result, a fall in blood oxygen saturation and/or a disruption in sleep may result.

CPAP therapy is a well-known and highly effective treatment for this condition. CPAP therapy typically uses a CPAP machine that delivers air to a CPAP mask that is attached to the CPAP machine.

A conventional CPAP mask includes a body that covers a user's nose or a user's nose and mouth, and which is held in place with one or more straps that are arranged over or about a user's head. The conventional CPAP mask also includes one or more hoses that connect the CPAP mask to the CPAP machine. CPAP users often find the conventional CPAP mask that covers their nose or nose and mouth (CPAP face mask), very uncomfortable. Many users have trouble with tightened straps that hold the conventional CPAP mask against the user's nose and/or mouth. An additional discomfort is the restriction to movement caused by the hose that attaches the conventional CPAP mask to the CPAP machine that is typically situated next to the user's bed.

In general, for many users the most uncomfortable aspect of the conventional CPAP mask is the feeling a user experiences when the user exhales. The user typically feels that he/she cannot exhale, i.e., cannot get their breath out. This feeling of incomplete breathing causes discomfort and results in many users (by some estimates 80% of users) becoming non-compliant (machine often not being used) within the first twelve months. Many users report that they feel that they sleep worse using the CPAP machine than without using the CPAP machine.

The condition that causes the need to use the CPAP machine is unforgiving, accumulative and ultimately results in significant health issues, and also lessens a user's quality of life. If users could tolerate the delivery of this therapy, they would benefit from it and avert the inevitable health issues associated with and common to non-treatment.

### SUMMARY

According to an aspect, a positive air way pressure mask includes a mask body having an inlet and an outlet, a nasal interface supported on the mask body, the nasal interface having air passages, with the nasal interface coupling a user's nose to the outlet of the mask body, and an air switch having an input port and an output port, with the input port configured to receive air from a continuous positive airway pressure machine and deliver the received air to the output port during a user inhalation, and further configured to at least partially block the air from the continuous positive airway pressure machine from passing from the input port to the output port during a user exhalation.

The above aspect may include amongst other features discussed herein one or more of the following features.

The mask further includes an exhalation valve having an inlet port that receives air from the outlet of the air switch, the exhalation valve having an outlet port that outputs air received from a user's nostrils, and further having at least one bi-directional port disposed in an airway that couples to the air passages in the nasal interface, with an exhalation opening the outlet ports when the user exhales to minimize air resistance during the user exhaling.

The mask further includes a pressure sensor that senses onset of a change in air pressure, the pressure sensor receiving air from a bi-directional port of the exhalation valve and producing in response a pressure signal, and electronic control circuit to control operation of the air switch in response to the pressure signal received from the pressure sensor.

The air switch is a voice coil actuated air switch that includes a switch body having a cavity, a first gasket, a second gasket, a plunger having a bulb portion and a shaft portion attached to the blub portion, a soft magnetic, iron pull piece attached to an end of the shaft portion, and a coil that causes the bulb portion to move into a passage in the first gasket to block air through the voice coil actuated air switch in a first mode and that causes the bulb portion to move away from the passage in the first gasket and into the second gasket to allow air to enter the voice coil actuated air switch and exit through output port of the air switch.

The voice coil actuated air switch includes a permanent magnet disposed within a region defined by the coil; and a magnetic iron shell disposed around the coil and permanent magnet.

The voice coil actuated air switch includes a permanent magnet disposed within a region defined by the coil.

The mask has a strap that holds the body against a user's face.

The electronic control circuit includes a microcontroller.

The microcontroller receives signals corresponding to changes in capacitance from the pressure sensor and converts the changes in capacitance to a pressure value.

The mask further includes a hose that couples the input port of the voice coil actuated air switch to an air outlet of the positive airway pressure machine.

The mask further includes a hose that couples the output port of the voice coil actuated air switch to the inlet of the mask body.

The mask further includes a first hose that couples the input port of the voice coil actuated air switch to an air outlet of the positive airway pressure machine and a second hose that couples the output port of the voice coil actuated air switch to the air outlet of the mask body.

The electronic control circuit includes a processor device, memory coupled to the processor device, an analog to digital converter to convert signals from the pressure sensor into digital signals, and storage storing computer instructions to cause the electronic control circuit to detect trigger points in a user's breathing.

The trigger points include at least one of an end of the user's inhalation and an end of the user's exhalation.

When electronic control circuit detects the end of the user's inhalation, the electronic control circuit is configured to set up for an exhalation portion of the user's breathing, initializes a delay timer, and generates after a time delay from the delay timer, a signal to control operation of a solenoid that controls operation of the air switch to divert air from the positive airway pressure machine to ambient.

When electronic control circuit detects the end of the user's exhalation, the electronic control circuit is configured to set up for an inhalation portion of the user's breathing, by initializing a delay timer, and generating after a time delay from the delay timer, a control signal to control operation of a solenoid controls operation of the voice coil actuated air switch to couple the air from the positive airway pressure machine to the inlet of the mask body.

The mask body houses the nasal interface, the air switch, and the exhalation valve.

The mask body houses the nasal interface, the voice coil actuated air switch, and the exhalation valve.

The mask body houses the nasal interface, the exhalation valve and the pressure sensor.

The mask body houses the nasal interface, the voice coil actuated air switch, the exhalation valve and the pressure sensor.

The mask body houses the nasal interface and the exhalation valve and wherein the pressure sensor is external to the mask body.

The mask body houses the nasal interface, the air switch, and the exhalation valve and wherein the pressure sensor is external to the mask body.

The mask body houses the nasal interface and the exhalation valve and wherein the voice coil actuated air switch and pressure sensor are external to the mask body.

According to an additional aspect, a continuous positive air way pressure mask arrangement includes a mask body having an inlet and an outlet, a nasal interface supported on the mask body, the nasal interface having air passages, with the nasal interface coupling a user's nose to the outlet of the mask body, and an air switch having an input port, an output port, and a diversion port, with the input port configurable to receive air from a continuous positive airway pressure machine and deliver the received air to the inlet of the mask body during an inhalation, and which switches the air from the positive airway pressure machine from entering the inlet port of the mask body to the diversion port diverting the received air to ambient during an exhalation.

The above aspect may include amongst other features discussed herein one or more of the following features.

The mask further includes an exhalation valve having an inlet port that receives air from the air switch output port, the exhalation valve having an outlet port that outputs air received from a user's nostrils, and further having at least one bi-directional port disposed in an airway that couples to the air passages in the nasal interface, with exhaling opening the outlet ports when the user exhales to minimize air resistance during the user exhaling.

The mask further includes a pressure sensor that senses onset of a change in air pressure, the pressure sensor receiving air from the at least one bi-directional port of the exhalation valve, and producing in response a pressure signal and electronic control circuit to control operation of the air switch in response to the pressure signal received from the pressure sensor.

The air switch includes a switch body having a cavity with a generally semicircular portion and a rotatable diversion element that forms part of a first and/or a second passage through the air switch and that is disposed in the semicircular portion and that has a channel that forms an air flow path with either the first passage or the second passage.

The air switch further includes a solenoid that is disposed to rotate the rotatable diversion element to complete or partially complete the first passage or the second passage, mode of operation of the air switch. The rotatable diversion element has a channel that forms an air flow path with either the first passage or the second passage.

The body has a strap that holds the body against a user's face.

The electronic control circuit includes a microcontroller. The microcontroller receives signals corresponding to changes in capacitance from the pressure sensor and converts the changes in capacitance to a pressure value.

The mask includes a hose that couples the air switch inlet to an air outlet of the positive airway pressure machine. The mask includes a hose that couples the air switch outlet to the air inlet of the positive airway pressure mask body.

The mask further includes a first hose that couples the air switch inlet to an air outlet of the positive airway pressure machine and a second hose that couples the air switch outlet to the air outlet of the positive airway pressure mask body.

The electronic control circuit includes a processor device, memory coupled to the processor device, an analog to digital converter to convert signals from the pressure sensor into digital signals, and storage storing computer instructions to cause the electronic control circuit to detect trigger points in a user's breathing. The trigger points comprise at least one of an end of the user's inhalation and an end of the user's exhalation. When the electronic control circuit detects the end of the user's inhalation, the electronic control circuit is configured to set up for an exhalation portion of the user's breathing, initializes a delay timer, and generates after a time delay from the delay timer, a signal to control operation of a solenoid that controls operation of the air switch to divert air from the positive airway pressure machine to ambient.

When the electronic control circuit detects the end of the user's exhalation, the electronic control circuit is configured to set up for an inhalation portion of the user's breathing, initializes a delay timer, and generates after a time delay from the delay timer, a control signal to control operation of a solenoid controls operation of the air switch to couple the air from the positive airway pressure machine to the air inlet of the positive airway pressure mask body.

The mask body houses the nasal interface and the exhalation valve. The mask body houses the nasal interface, the air switch, and the exhalation valve. The mask body houses the nasal interface, the exhalation valve and the pressure sensor. The mask body houses the nasal interface, the air switch, the exhalation valve and the pressure sensor. The body houses the nasal interface and the exhalation valve and wherein the pressure sensor is external to the mask body. The mask body houses the nasal interface, the air switch, and the exhalation valve and wherein the pressure sensor is external to the mask body. The mask body houses the nasal interface and the exhalation valve and wherein the air switch and pressure sensor are external to the mask body.

Aspects also include computer program products and methods.

One or more of the above aspects may provide one or more of the following advantages.

Unlike the conventional CPAP mask, the discomfort when the user exhales is substantially eliminated. The user typically feels that he/she can easily exhale, i.e., get their breath out because of the air switch. The air switch device switches air received from a continuous positive airway pressure machine to the input of the CPAP mask during a user inhaling and switches air to divert to ambient or block some or all of the incoming air when a user exhales.

In addition, the exhalation valve receives air at the inlet port of the exhalation valve when the user inhales and opens the outlet ports when the user exhales and therefore minimize air resistance during the user exhaling.

The air switch (solenoid and/or voice coil actuated) and exhalation valve virtually eliminate the feeling of incomplete breathing caused by the conventional CPAP mask, which occurs when the user exhales against an inrush of air from the CPAP machine. Elimination of this feeling should result in many more users becoming machine compliant (machine often used). More regular use of the CPAP machine will ultimately lead to minimizing occurrence of significant health issues, caused by users abandoning treatment by the CPAP machine.

The details of one or more embodiments of the invention are set forth in the accompanying drawings and the description below. Other features, objects, and advantages of the invention are apparent from the description and drawings, and from the claims.

### DESCRIPTION OF DRAWINGS

FIG. 1 is a diagram depicting a continuous positive airway pressure mask and associated components coupled to a continuous positive airway pressure machine.

FIG. 2 is a block diagram showing components in the mask of FIG. 1.

FIGS. 2A and 2B are diagrams showing connection of pressure sensor(s) to an exhalation valve.

FIGS. 3A-3D are diagrammatical views of an air switch.

FIG. 4 is a flow diagram.

FIG. 4A is a graph of pressure vs. time.

FIG. 5 is a block diagram showing an alternative arrangement of components in a CPAP mask similar to that of FIG. 1.

FIGS. 6A-6D are diagrams depicting another example of a CPAP mask body.

FIGS. 6E-6G are diagrams depicting features of the CPAP mask body illustrated in FIGS. 6A-6D.

FIG. 7 is a block diagram of an electronic control circuit.

FIG. 7A is a block diagram showing an alternative CPAP mask with an alternative air switch.

FIGS. 8A-8E are diagrams of the alternative air switch.

### DETAILED DESCRIPTION

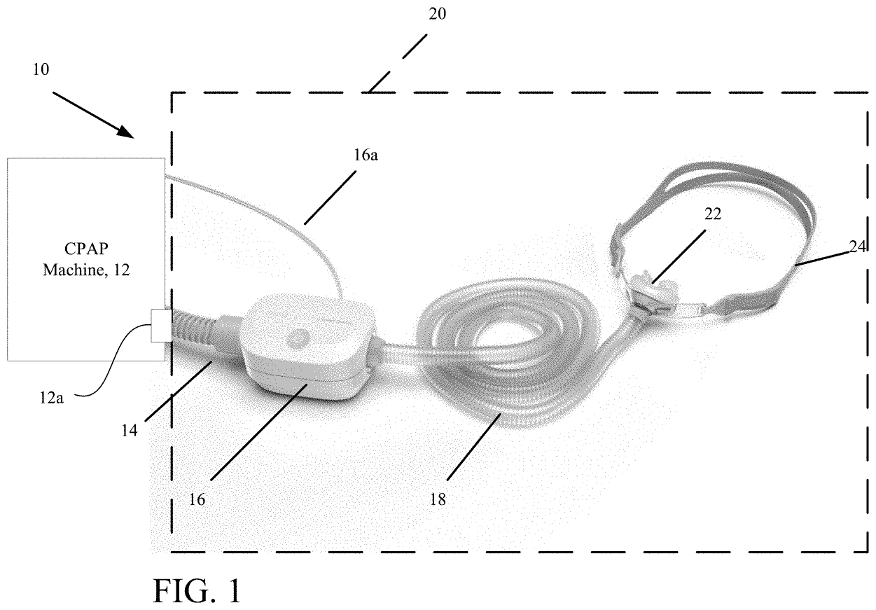

Referring to FIG. 1, continuous positive airway pressure (CPAP) system 10 includes a CPAP machine 12 that is coupled to a CPAP mask 20. The CPAP mask 20 has a low level of discomfort when the user inhales. The CPAP mask 20, in addition, also has a low level of discomfort when the user exhales, e.g., the level of discomfort is substantially eliminated, as will be described below.

The CPAP machine 12 has an air outlet 12a and is generally conventional and can be any of the CPAP machines available on the market. Non-limiting examples of CPAP machines include AirSense™ 10 or AirSense™ 11 CPAP and APAP or AirCurve™ 10 Bilevel machines from ResMed (San Diego, Calif.)1 Dream Station from Phillips Respironics (Murrysville PA). Other CPAP type machines can be used as well.

In general, with the CPAP mask 20 a user sets up the CPAP machine 12 according to a physician's orders. The CPAP mask 20 has circuitry that controls the delivery of positive air pressure from the CPAP machine 12 to, e.g., the user's nose when the user inhales and inhibits the airflow from reaching the user's nose when the user exhales. The CPAP system 10 has the CPAP machine 12 coupled to a first hose 14 having two ends. One end of the first hose 14 is coupled to the air outlet 12a of the CPAP machine 12 and the second end is coupled to an air inlet of an air switch stage 16 of the CPAP mask 20.

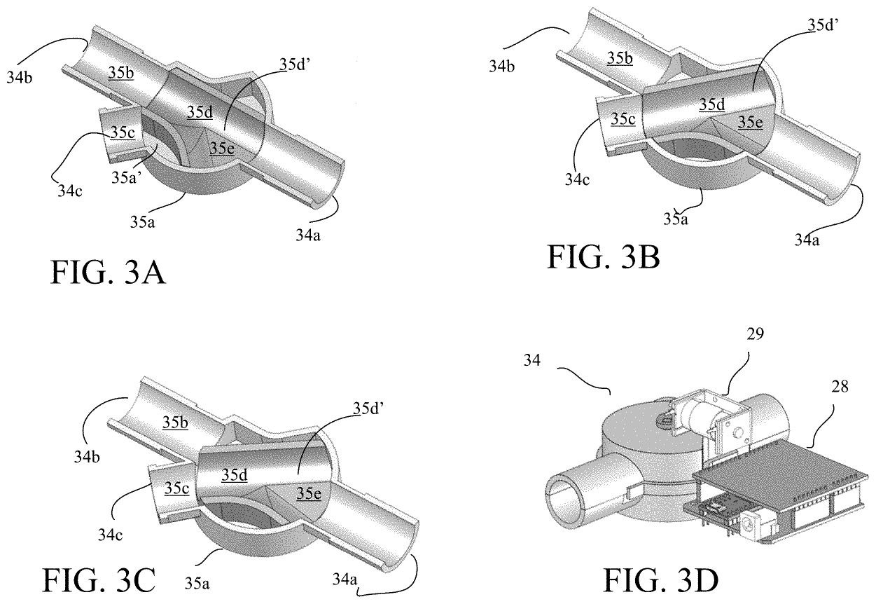

The air switch stage 16 is used to divert an inrush of air from entering the user's nose, as the user is attempting to exhale. Details on construction of an air switch are given in FIGS. 3A-3C. An air outlet of the air switch stage 16 is coupled to a second hose 18 having two ends, with one end coupled to the air outlet of the air switch stage 16 and the other end coupled to an air inlet of a CPAP mask body 22 of the CPAP mask 20. The CPAP mask 20 in addition to the CPAP mask body 22 generally has a strap 24 that is coupled to the CPAP mask body 22. The strap 24 is used to secure the CPAP mask body 22 of the CPAP mask 20 to a user's face and more particularly a user's nose. A line 16a can be used to deliver electrical power to the air switch stage 16.

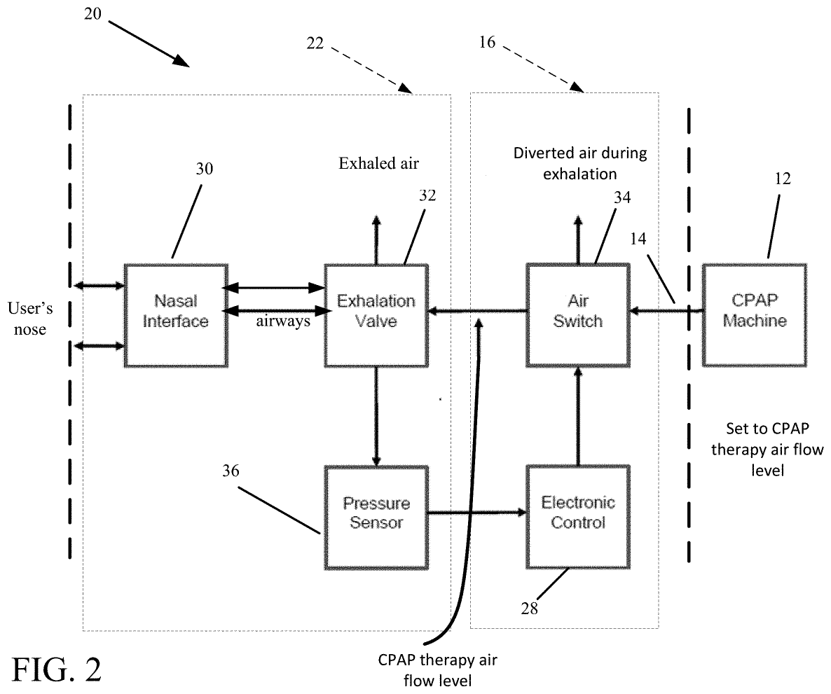

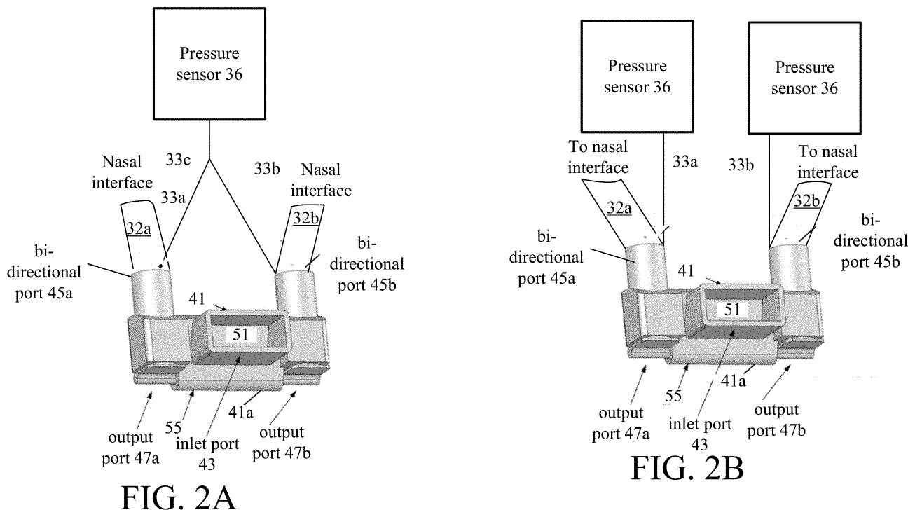

Referring now to FIG. 2, one embodiment of the CPAP mask 20 is shown. The CPAP mask 20, in addition to the CPAP mask body 22 and strap 24 (the strap is not shown in FIG. 2 for clarity, see FIG. 1.), includes a nasal interface 30, an exhalation valve 32, an air switch 34 and a pressure sensor 36. An electronic control circuit 28 is also included to control operation of the air switch stage 16 based, in part, on signals from the pressure sensor 36. The air switch 34 and the electronic control circuit 28 are shown as in the air switch stage 16. The pressure sensor 36 is used to track a user's breathing cycle, so as to indicate to the electronic control circuit 28 when to divert air from the CPAP machine 12 from entering the CPAP mask 20 and interfering with the exhale portion of the user's breathing cycle. The pressure sensor 36 senses a change in pressure from the user inhaling air and the user exhaling air, and sends these changes in pressure to the electronic control circuit 28 in the form of changes in capacitance that are expressed as electrical signals which can be analog or digital values.

The CPAP machine 12 is coupled to the air switch 34, via the first hose 14 (see FIG. 1). The air switch 34 is controlled by the electronic control circuit 28. Electronic control circuit 28 sends a control signal to the air switch 34 to cause the air switch 34 to divert incoming air from the CPAP machine 12 into an exhaust line (away from the user) during an exhalation portion of a user's breathing cycle and to switch air to an air outlet of the switch (to the user) during an inhalation portion of the user's breathing cycle. The exhalation valve 32 is a micro bi-directional valve that allows user to easily inhale air from the CPAP machine 12 and easily exhale air from the user's lungs into an ambient. Together with the air switch 34 it avoids the user battling an incoming rush of air from the CPAP machine 12 and allows the user to easily exhale air from the user's lungs directly into the ambient, without the exhaled air going back through the air switch 34.

Nasal Interface

One embodiment of the nasal interface 30 is disclosed in U.S. patent application Ser. No. 15/648,504, filed Jul. 13, 2017, published as: US-2018-0015247-A1 on Jan. 18, 2018, and entitled “Nasal Interface for CPAP Device,” the entire contents of which are incorporated herein by reference. Other types of nose buds could be used.

The nasal interface 30 are nose buds that are attached to CPAP mask body 22 of the CPAP mask 20. The nose buds have flare/flange fittings at end portions thereof, as described in the above patent application. The flare/flange fittings are configured for easy insertion and comfortable positioning inside nostril cavities to provide a sealing interface to a user's nostrils. The nose buds as described in the above application however were attached to a CPAP machine, and not to a CPAP mask 20. As described in the above application, the nasal interface 30 allows unobstructed breathing during both inhalation and exhalation in the described CPAP machine.

In the CPAP mask 20, the nose buds together with the other components, as described in FIG. 1, also allows for unobstructed breathing during both inhalation and exhalation. The flare/flange fittings are integrally provided (e.g., formed) on nose bud sidewalls, and are sized to allow the user to easily insert the nose buds into the user's nose, preferably with one hand, without excessive adjusting motion. The nose buds provide a seal between the CPAP mask body 22 and the user's nose. Within the flare/flange fittings are at least one aperture that permits air to flow through the nasal interface 30.

However, other more conventional nose buds may be used, such as traditional nose pillow type design, e.g., a simple nose pillow (Hershey® kiss shape) may be used. The nose buds referenced in the above application were conceived to keep the uCPAP machine in a user's nose without the need for straps. With the CPAP mask 20 described herein to connect to the CPAP machine 12 the use of a hose may necessitate use of the straps. Therefore, because straps may be needed, the simple nose pillow (Hershey® kiss shape) may be better. However, neither design changes the basic functional operation of the CPAP mask 20.

Exhalation Valve

One embodiment of the exhalation valve 32 is disclosed in U.S. patent application Ser. No. 16/242,083, filed Jan. 18, 2019, published as: US-2019-0209797-A1 on Jul. 11, 2018, and entitled “Micro bi-Directional Valves and Systems,” the entire contents of which are incorporated herein by reference.

The exhalation valve 32 has a bi-directional valve configuration. The exhalation valve has a paddle mechanism that uses air flow from the CPAP machine 12, via the air switch 34, to open/close passages in the exhalation valve 32 at the end of an exhalation/beginning of pause in breathing and at the beginning of exhalation. The exhalation valve as described in the above application however was attached to a CPAP device, and not to a CPAP mask 20.

The exhalation valve 32 including a body, a single unidirectional port that is used as an inlet, a pair of bi-directional ports, and a pair of unidirectional ports that are used as outlet ports. Each of the outlet ports has a paddle that selectively closes and opens the respective port. The inlet port also has a paddle. The paddles are flat members and are part of paddle valve mechanism. The paddle valve mechanism is rotatable within an axial compartment (or airway) provided in the body at a body portion to open and close passageways among ports, as described and illustrated by FIGS. 3A-31 of the above incorporated by reference patent application.

The pair of bi-directional ports of the exhalation valve 32 are coupled to the inlet of the pressure sensor 36. The pair of bi-directional ports of the exhalation valve 32 are coupled together by a “Y” configuration of a pair of hoses that feed a common hose that is attached to the inlet of the pressure sensor 36.

Referring now to FIGS. 2A and 2B, a figure (FIG. 3A) that is adapted from the '083 patent application, is shown with the exhalation valve 32 including a pair of bi-directional ports 45a, 45b, a pair of outlet ports 47a, 47b and an input port 43.

FIG. 2A shows the figure (FIG. 3A) from the '083 patent application adapted to include hose sections 32a, 32b, which couple the bi-directional ports to the nasal interface 30 and a Y configuration of hose sections 33a, 33b and 33c. Hose sections 33a and 33b have one end that is attached to the bi-directional ports 45a, 45b of the exhalation valve 32, and have another end that is attached together with an end of the hose section 33c. The other end of hose section 33c is an outlet of the Y connection of hose sections 33a-33c, and which is coupled to the inlet of the pressure sensor 36.

FIG. 2B shows the figure (FIG. 3A) from the '083 patent application adapted to include hose sections 32a, 32b, i.e., second hose 18 FIG. 1, and the pair of hose sections 33a, 33b attached to the bi-directional ports 45a, 45b of the exhalation valve 32 and inlets of a pair of the pressure sensors 36.

In some embodiments a second pair of pressure sensors can be coupled, via hose sections, to (each nostril), bi-directional ports 45a or 45b, which have a perpendicular sense point and an inline sense point. Specifically, using the configuration of a perpendicular sense point along with an inline sense point, in an airflow channel (like 45a for example), allows for more complex analysis of air flow and pressure, as opposed to just using one sense point. Alternatively, sensing could occur at the output ports or the input port, if desired.

In each of these configurations, the pressure sensor(s) monitor air pressure changes to cause the electronic control to switch the air switch 34, as discussed below.

Pressure Sensor

One embodiment of the pressure sensor 36 is disclosed in U.S. patent application Ser. No. 15/668,837, filed Aug. 4, 2017, published as: US-2018-0038754-A1 on Feb. 4, 2018, and entitled “Micro Pressure Sensor,” the entire contents of which are incorporated herein by reference.

The pressure sensor 36 includes a body having a first pair of opposing walls and a second pair of opposing walls that are orthogonal to the first pair of opposing walls and that define a chamber. A plurality of membranes each having a corresponding electrode layer over a surface thereof are disposed in the chamber and anchored between the first pair of opposing walls of the body to provide plural compartments within the chamber. A first set of ports is coupled to a first set of the plural compartments. The first set of ports are disposed in corresponding portions of a first one of the first pair of opposing walls of the body, with a second one of the first pair of opposing walls of the body being a solid portion of the body. A second set of ports are coupled to a second different set of the plural compartments, with the second set of ports disposed in corresponding portions of the second one of the first pair of opposing walls of the body, with the first one of the first pair of walls of the body being a solid portion of the body, as described and illustrated by FIGS. 1-7 of the above incorporated by reference patent application.

The pressure sensor 36 outputs an electrical signal that is indicative of pressure changes as mentioned in the above incorporated by reference patent application. The electrical signals can be carried from the pressure sensor by a set of wires that are integrated within the second hose 18 from the electronic control circuit 28 that includes the microcontroller 28a and a voltage source 28g.

Air Switch

Referring now to FIGS. 3A-3C, which show the air switch 34 in various stages of operation. The air switch 34 comprises a body 35a having three ports 34a-34c. In FIGS. 3A-3C, inlet port 34a is coupled to the CPAP machine 12, outlet port 34b is coupled to an input port 43 of the exhalation valve 32, and diversion port 34c is coupled to the ambient. Inlet port 34a and outlet port 34b are coupled via a first passage 35b and inlet port 34a and diversion port 34c are coupled by a second passage 35c. Both the first and second passages have paths through the body 35 that are curved and that match with paths in a cover portion of the body 35.

The body 35a has an cavity 35a′ with a semicircular portion that accommodates rotation of a rotatable diversion passage 35d that forms part of the first passage 35b and/or second passage 35c. Disposed in the first passage 35b and the second passage 35c is the rotatable diversion passage 35d that rotates into positions to completely or partially complete the first passage 35b and/or the second passage 35c, according to the mode of operation of the air switch 34.

First passage 35b and second passage 35c and rotatable diversion passage 35d are shown generally semi-circular in shape. The rotatable diversion passage 35d forms an input air flow path with either the first passage 35b and/or the second passage 35c. The rotatable diversion passage 35d at least partially blocks air at the inlet port 34a from reaching the outlet port 34b, when the air switch is configured to divert a portion of air from the inlet port 34a to the diversion port 34c. A device or mechanism could be used to divert a portion of the air. The mechanism could be a “separate” element or an adjustment to the length of solenoid travel. For dynamic control a motor could be used that would be driven a position required to produce the “leak” (FIG. 3C) sufficient to allow a minimum air flow.

FIG. 3A shows the air switch 34 in an open state. The open state allows input air from the CPAP machine 12 to freely pass through the first passage 35b connecting inlet port 34a and outlet port 34b. In the open state, the rotatable diversion passage 35d is positioned such that the rotatable diversion passage 35d is in alignment with the first passage 35b, with a sidewall of the rotatable diversion passage 35d blocking the second passage 35c.

FIG. 3B shows the air switch 34 in a diversion state. The diversion state allows input air to freely pass through the second passage 35c connecting inlet port 34a and diversion port 34c. In the diversion state, the rotatable diversion passage 35d is positioned such that the rotatable diversion passage 35d is in alignment with the second passage 35c, with a sidewall of the rotatable diversion passage 35d blocking the first passage 35b.

FIG. 3C shows the air switch 34 in a partial diversion state. The partial diversion state allows a first portion of input air to pass through the first passage 35b connecting inlet port 34a and outlet port 34b and a remaining, second portion of air to pass through the second passage 35c connecting inlet port 34a and diversion port 34c. The partial diversion state is adjustable. Generally, the first portion of the input air to pass is 5% to 20% and the second portion of the input air to pass is 95% to 80%. Other ranges are possible. In the partial diversion state, the rotatable diversion passage 35d is positioned such that the rotatable diversion passage 35d is in somewhat alignment with the second passage 35c, with a sidewall of the rotatable diversion passage 35d at least partially blocking air into the first passage 35b.

FIG. 3D shows the air switch 34 packaged with a cover portion 34d. The cover portion 34d in this embodiment has corresponding features as the first passage 35b and second passage 35c and the rotatable diversion passage 35d. The air switch is also packaged with the electronic control circuit 28 that sends signals to control operation of a solenoid 29. The solenoid 29 has a mechanism that allows the solenoid 29 to rotate the rotatable diversion passage 35d to either divert air to the diversion port 34c of the air switch 34 and/or to the outlet port 34b of the air switch 34 by controlling movement of the rotatable diversion passage 35d. In some embodiments, a motor could be used instead of the solenoid 29 to provide more precise control of the movement of the rotatable diversion passage 35d, if desired.

One such motor is described in U.S. patent application Ser. No. 16/418,254, filed May 21, 2019 entitled “Micro Electrostatic Motor and Micro Mechanical Force Transfer Devices,” the entire contents of which are incorporated herein by reference.

In some implementations, the air switch 34 can be integrated within the CPAP mask body 22 along with the solenoid 29 and electronic control circuit 28.

Pressure Monitoring

Pressure monitoring—There are four ports capable of monitoring air pressure inside the body piece. Each nostril has one port that is in-line with the airflow through the nostril and one port that is perpendicular to this airflow. This arrangement of sense points allows more complex analysis of pressures and flows within the body piece, right at the nose.

In the implementation of FIG. 1, one pressure sensor 36 is monitoring the inline ports of both nostrils. These ports are plumbed together in a “Y” fashion and fed to the pressure sensor 36. In other implementations, there can be up to four independent pressure sensors, one for each port monitoring the patient's breathing.

Control Process

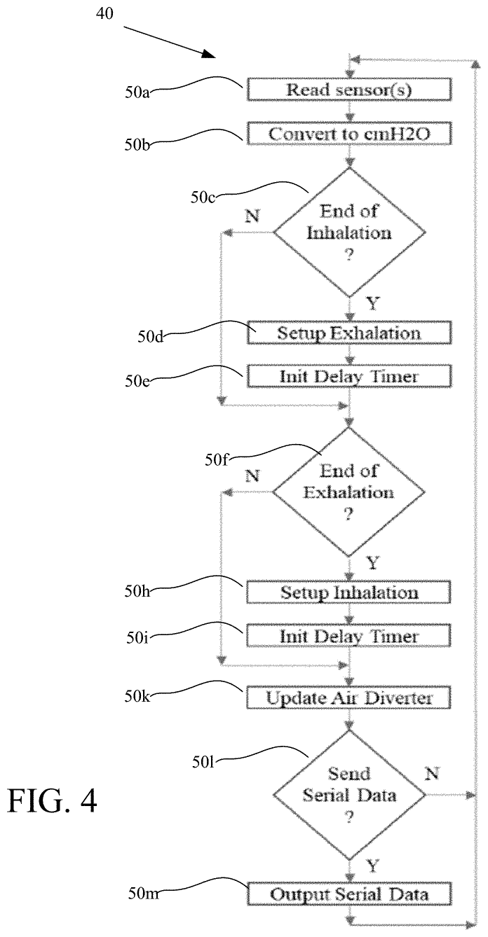

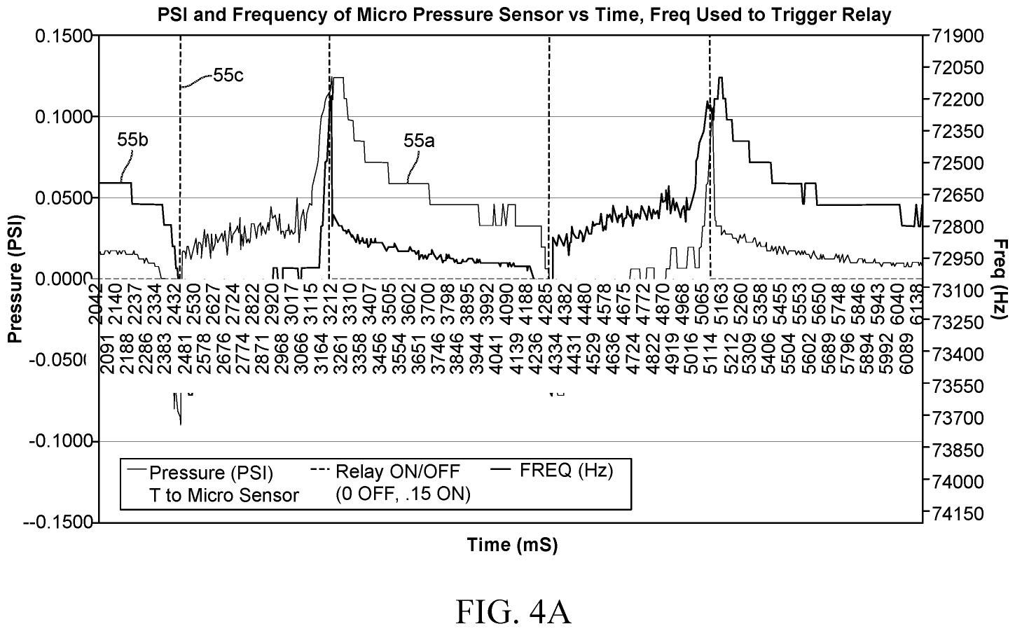

Referring now to FIG. 4, a control process 50 for the electronic control circuit 28 is shown. For a patient who is wearing the CPAP mask body 22 properly connected to their CPAP machine 12, the pressure sensor 36 senses pressure changes in the patient's breathing The control process 50 analyzes the patient's sensed pressure changes in breathing from the pressure sensor 36 to control the CPAP airflow from the CPAP machine 12 to the CPAP mask 20.

Control process 50 has a controller reading 50a pressure data from the pressure sensor 36. Control process 50 converts 50b the pressure data from the pressure sensor into a pressure measured as cmH₂O (center meters of water). However, other measures of pressure could be used. Control process 50 searches 50c for an end of inhalation. When control process 50 indicates an end to inhalation, the control process 50 sets up for an exhalation 50d, by insuring the air switch 34 couples air from the inlet port 34a to the outlet port 34b and initiates a delay timer 50e. Delay timer 50e introduces a delay in transitioning from inhalation to exhalation.

Control process 50 searches 50f for an end of exhalation. When control process 50 indicates an end to exhalation, the control process 50 sets up for an inhalation 50h initiates a delay timer 50i, and sends 50g a signal to the air switch 34 to couple air from the inlet port 34a to the outlet port 34b of the switch. Delay timer 50i introduces a delay in transitioning from exhalation to inhalation prior to diverting air via the air switch 34.

In some embodiments, the control process 50 can send 501 serial data and output 50m serial data to a monitor. The control process 50 can be used to build a patient's breathing waveform profile. The control process 50 uses identified inflection points that correspond to certain physiological actions, such as inspiration, expiration and pause. Using pattern recognition principles or simple trigger points, thresholds can be developed to identify transition points in the patient's breathing.

In the implementation of FIG. 2, the control process 50 looks for proper transition points that indicate the end of an inhalation and/or the start of an exhalation, and the end of an exhalation and/or start of an inhalation.

When the control process 50 identifies the start of an exhalation (as indicated by the proper threshold) the control process 50 activates the solenoid 29 which orients the air switch mechanism to divert air, or some portion of the air from the CPAP machine 12, away from the patient. The air is diverted during the majority of the exhalation and until the start of an inhalation is indicated. At this point, the air diverter mechanism returns to its default position which supplies the CPAP airflow to the patient for the duration of their inhalation. This process repeats for every breath as it tracks the patients breathing.

The control process 50 looks for specific thresholds to be indicated. When the patients breathing slows or changes, the point at which air diversion happens will also change. This control process 50 is not running at a pre-timed open loop control. Rather, the control process 50 follows the patients breathing.

While FIG. 4 shows a simple flow chart of the control process 50, additional routines may be used to establish additional dynamic thresholds that can be adjusted, identified and logged, such as apnea and hypopnea events. Also other events can be logged, e.g., stored such as CPAP machine 12 use time, sleep duration, as well as other functions. This data can be stored and retrieved for further processing and analysis.

The CPAP mask 20 can include some or all of the following additional routines, such as determining the patient's treatment level of pressure coming from the CPAP machine 12 and to determine when an apnea event is occurs. The CPAP mask 20 can count a number of apnea events. The CPAP mask 20 determines how long the CPAP machine 12 is being used to determine a user compliance. The CPAP mask 20 can auto titrate down treatment level pressure, e.g., lower treatment level, when a lower level of treatment is needed. The CPAP mask 20 can produce various stimuli during an apnea event, e.g., to shorten length of apnea event. The CPAP mask 20 can adjust inhalation and exhalation thresholds for maximum comfort and store sleep data for subsequent retrieval.

FIG. 4A shows a graph of two cycles of a patient's breathing. The pressure 55a and frequency curves 55b represent the breathing pressure and patient's timing used to generate the control signal 55c to control the solenoid to divert air. The solenoid can be seen to transition at the thresholds of inhalation (goes high) and exhalation (goes low).

Alternative Pressure Sensor

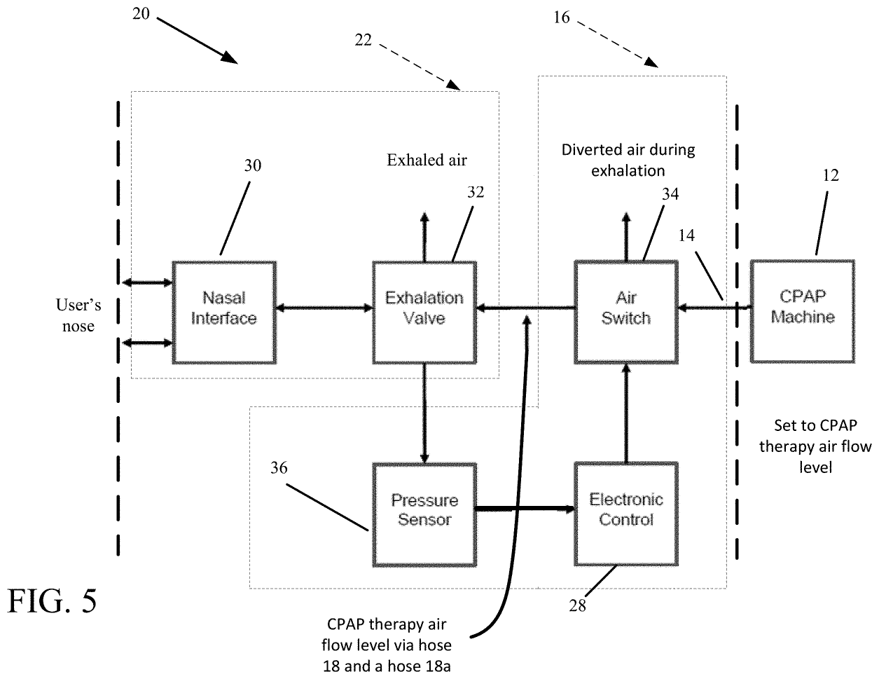

Referring now to FIG. 5, an alternative CPAP mask 20′ uses alternative embodiments of a pressure sensor, rather than the pressure sensor 36 of FIG. 2. For example, pressure sensors that are not of the pressure sensor 36 dimensions and thus would not fit in the CPAP mask 20 could still be used. Instead, such pressure sensors would be located in or adjacent to the air switch stage 16 along with the electronic control circuit 28 (or possible within the CPAP machine 12). However, the pressure sensor still needs to sense pressure at a user's nose. In order to sense pressure, a pressure sensor input would be coupled to the bi-directional ports of the exhalation valve 32 by a smaller diameter hose 18a that could be disposed within the second hose 18, which is a larger diameter hose.

In some embodiments, both outlets of the exhalation valve 32 are fed by air being exhaled from a user's nose. One or both of these outlets could be coupled to the inlet of the pressure sensor. In one embodiment, the pair of outlets are coupled to the small hose via the “Y” connection of hoses, as mentioned above.

One example of such a pressure sensor is the TruStability® Standard Accuracy Silicon Ceramic (SSC) Series Board Mount Pressure Sensors. The TruStability® is a piezo-resistive silicon pressure sensor offering a ratio-metric analog or digital output for reading pressure over the specified full scale pressure span and temperature range, available from Honeywell. This is merely one example, other examples could be used.

CPAP Body Mask

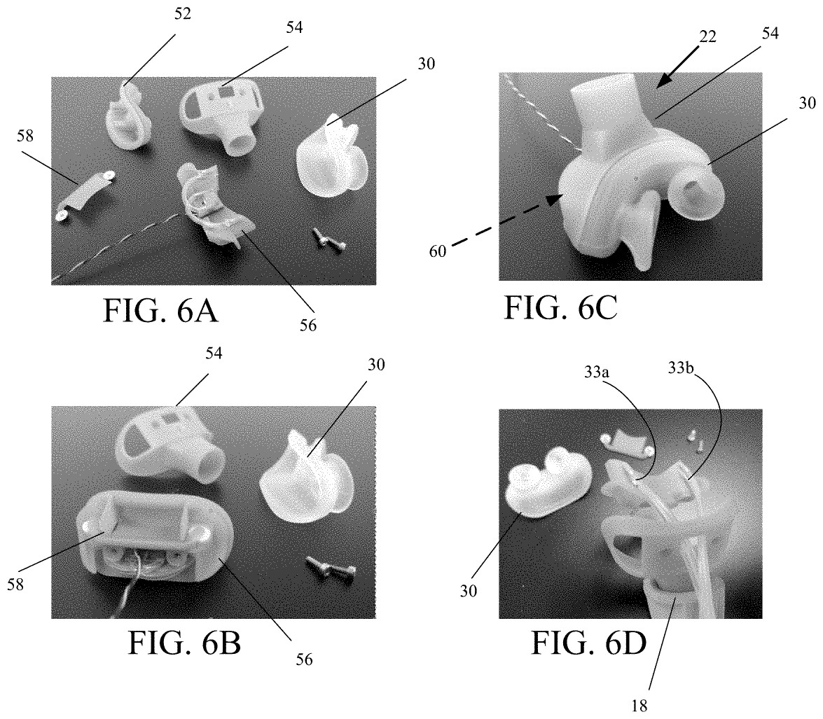

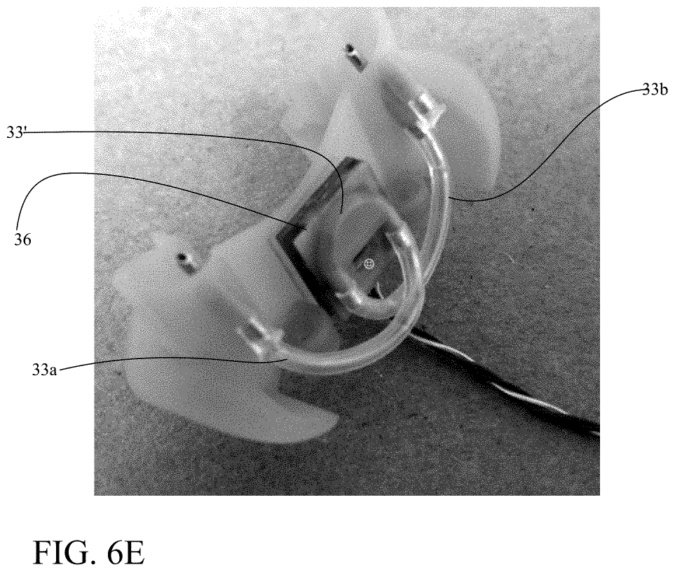

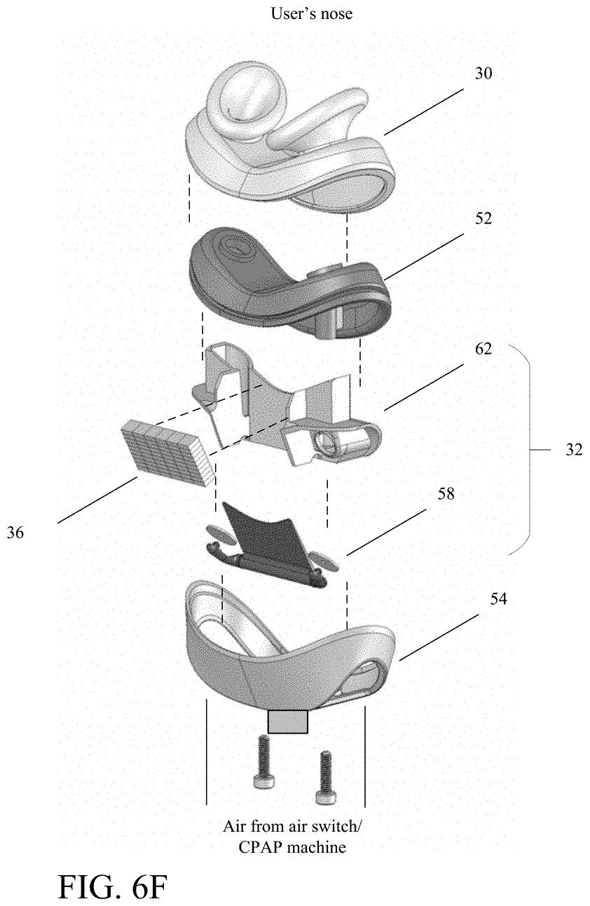

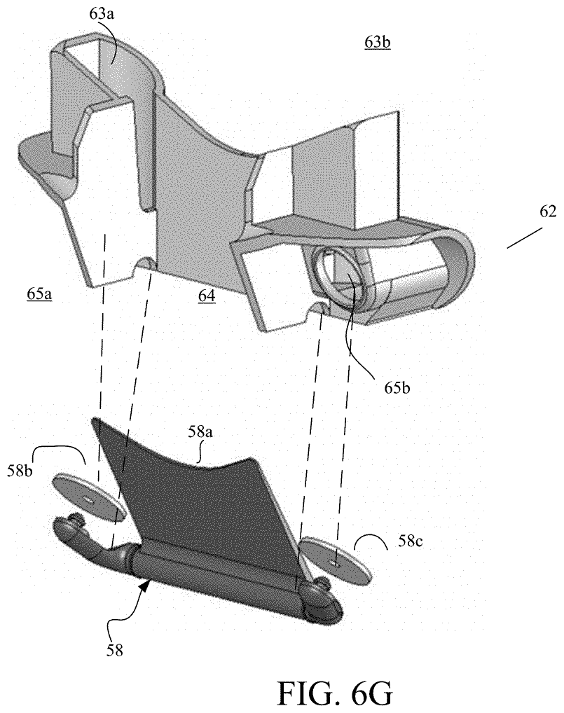

Referring now to FIGS. 6A-6D example of the CPAP mask body 22 is shown. FIG. 6A includes component parts that will be integrated into the CPAP mask body 22. These component parts include a nasal interface receiver 52 that receives the nasal interface 30. The component parts also includes a hose receiver 54 that receives the second hose 18 (shown in FIGS. 1, 6D) a pressure sensor holder 56 that houses the pressure sensor 36 and a paddle mechanism 58 that in combination with the aforementioned components forms a functional exhalation valve of the type shown in FIGS. 2A-2B.

FIG. 6B shows the nasal interface 20 and the hose receiver 54 and the paddle mechanism 58 secured to the pressure sensor holder 56.

FIG. 6C shows the CPAP mask body 22. The CPAP mask body 22 has the nasal interface receiver 52 attached to the nasal interface 30. The nasal interface receiver 52 is also attached to the combination 60 of the paddle mechanism 58 and the pressure sensor holder 56 (indicated by a phantom arrow). The hose receiver 54 (second hose 18 shown in FIG. 6D) is attached to the other side of the combination 60 of the paddle mechanism 58 and the pressure sensor holder 56.

FIG. 6D shows the second hose 18 attached to the hose receiver 54 and shows hose section 33a and hose section 33b (see FIG. 2A) attached to the pressure sensor holder 56. Attachment to the pressure sensor body effectively completes the Y connection by effectively providing the hose section 33c (see FIG. 2A) to the pressure sensor 36.

Referring now to FIGS. 6E-6G details of the CPAP mask body 22 are shown.

FIG. 6E shows details of the pressure sensor holder 56. The pressure sensor holder 56 is shown with hose section 33a and the hose section 33b attached to receptacles with the hose section 33a and the hose section 33b having a join 33′ that is attached to a pressure inlet of the pressure sensor 36.

FIG. 6F shows the CPAP mask body 22 in an exploded view, with the nasal interface 30 over the nasal interface receiver 52 that attaches to the pressure sensor holder 56. Disposed under the pressure sensor holder 56 is an airway passage 62 that is effectively part of the exhalation valve 32 (FIGS. 2 and 2A) and the pressure sensor 36. The paddle mechanism 58 is secured to the airway passage 62 and together with the paddle mechanism 58 in effect provides the exhalation valve 32. The hose receiver 54 is secured to the nasal interface 30 via a pair of screws or other types of fasteners, such as rivets, pins, clips, etc. or may be glued, fused, etc.

FIG. 6G shows the airway passage 62 and the paddle mechanism 58 that in effect provides the exhalation valve 32 of FIG. 2. Bi-directional ports 63a, 63b (effective positon) are shown as are the inlet port 64 (effective positon), and outlet ports 65a (effective positon), 65b. The paddle mechanism 58 has a center paddle 58a and side paddles 58b, 58c. The center paddle 58a and the side paddles 58b, 58c close the outlet ports 65a, 65b during inhalation.

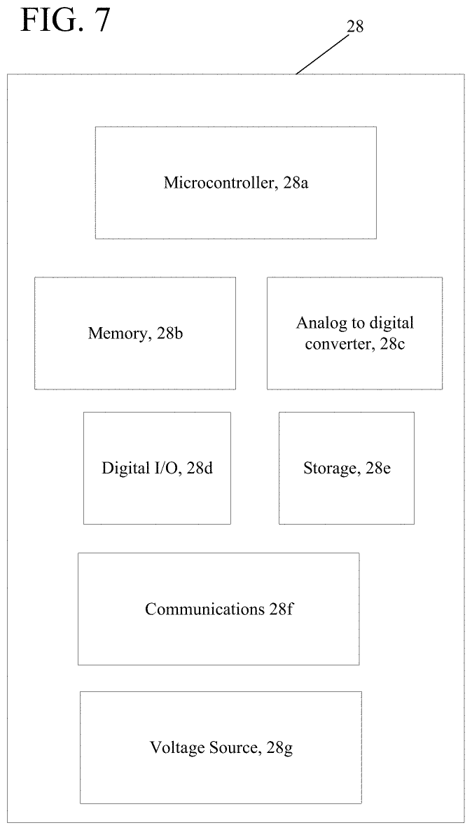

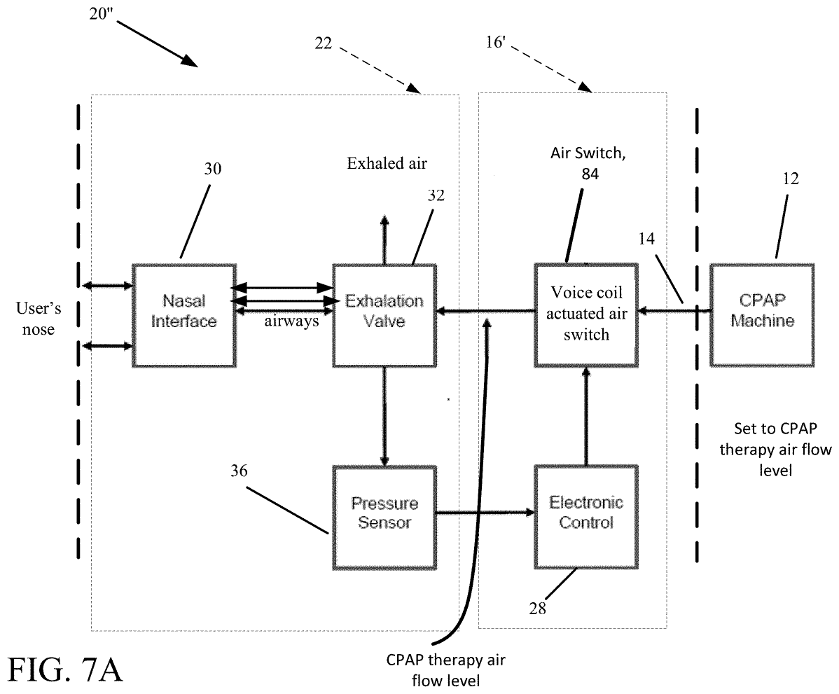

FIG. 7 shows the electronic control circuit 28 as including a microcontroller 28a, memory 28b, an analog to digital converter 28c, I/O (input/output) 28d, storage 28e, communications 28f and a voltage source 28g. These are functionally coupled together to control the pressure sensor(s) 26 and perform the functions illustrated in FIG. 4.

Alternative Air Switch

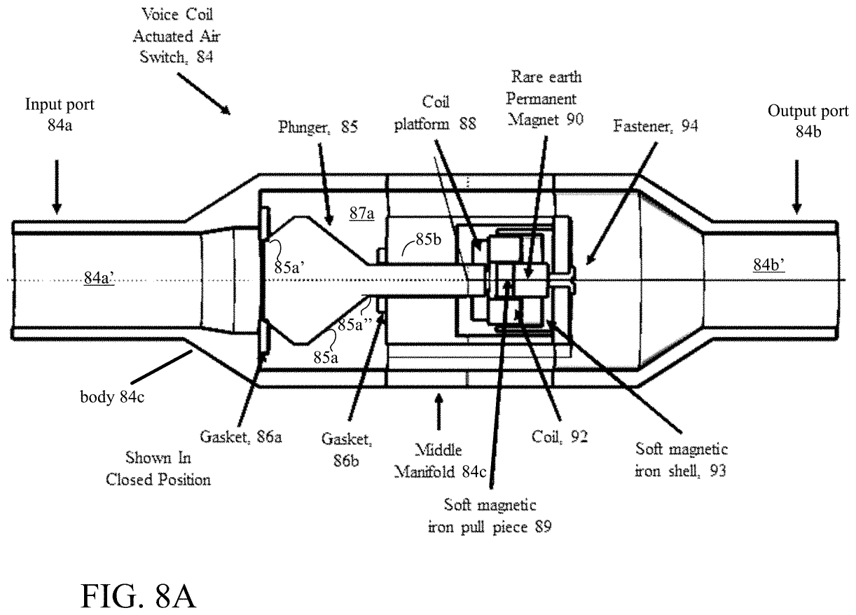

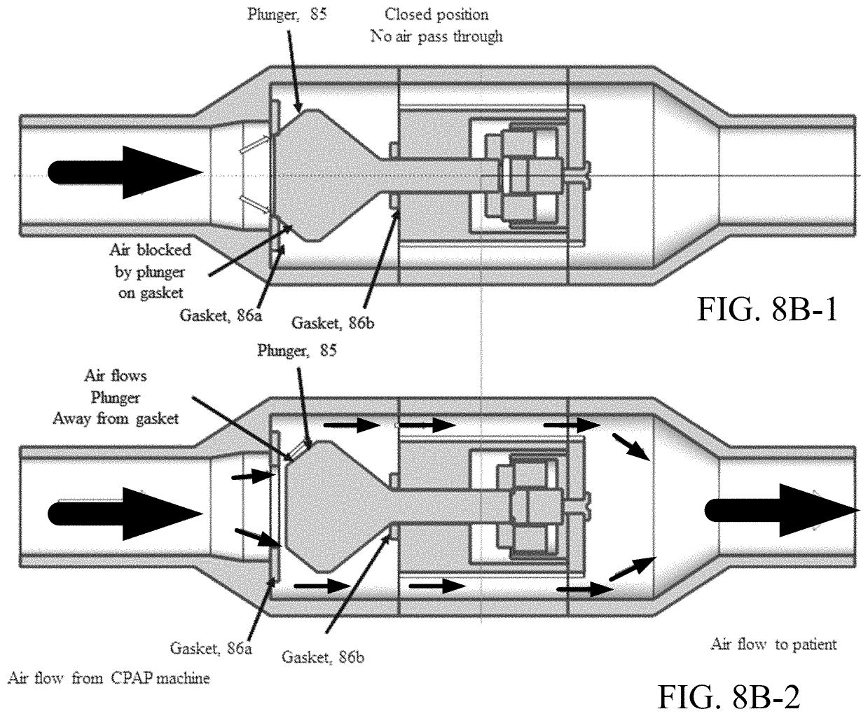

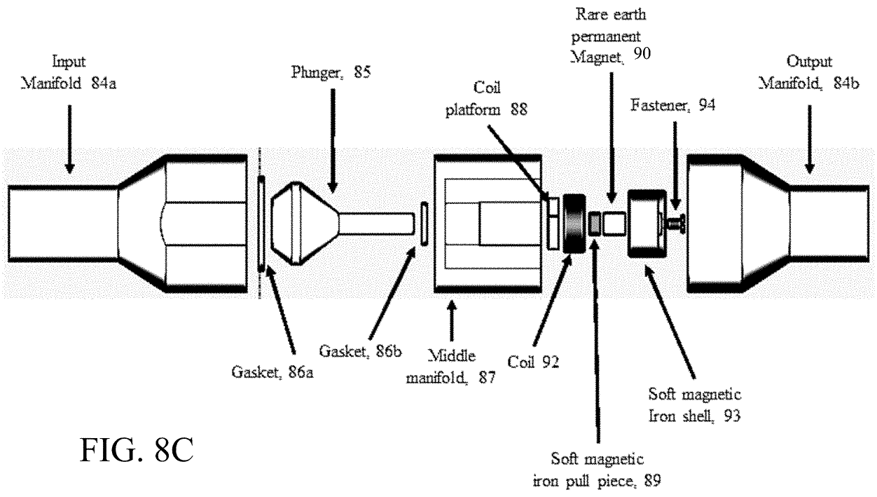

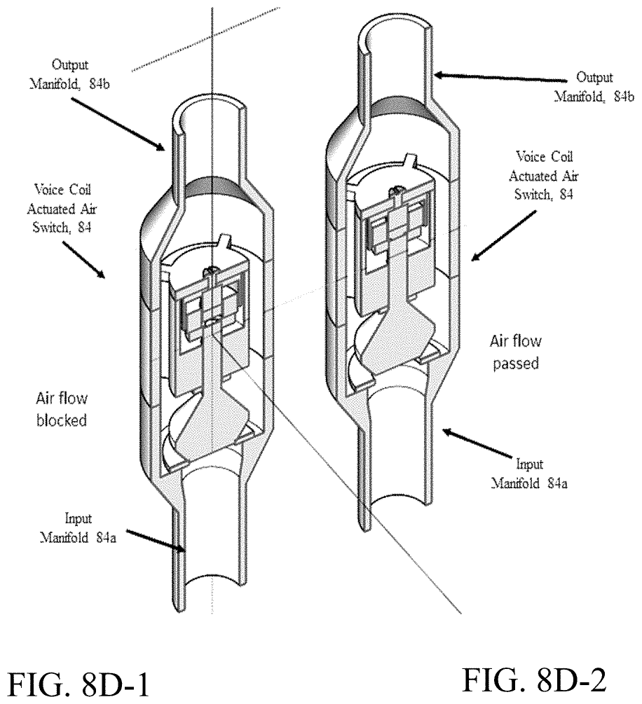

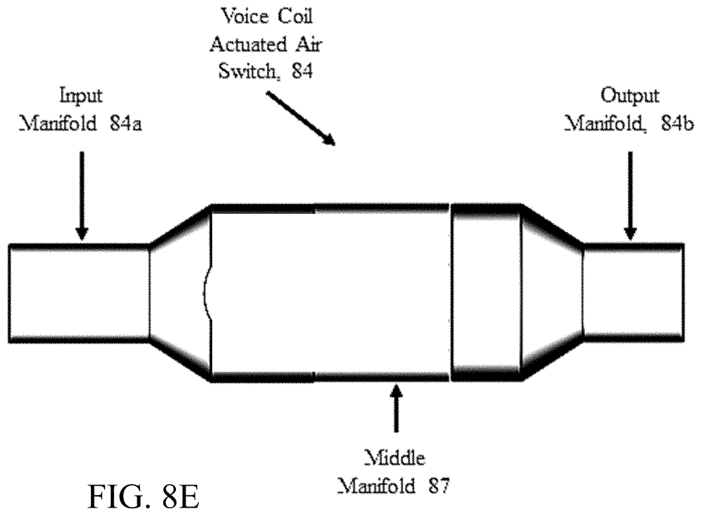

Referring now to FIG. 7A, another alternative CPAP mask 20″ is shown. The CPAP mask 20″ includes the CPAP mask body 22 and may include a strap 24 (the strap is not shown in FIG. 7A for clarity, but see FIG. 1.), the nasal interface 30, the exhalation valve 32, and the pressure sensor 36. The alternative embodiment also includes an alternative air switch, i.e., a coil, or a so called “voice coil,” actuated air switch (air switch) 84, see FIGS. 8A-8E for details. The air switch 84 is controlled by the electronic control circuit 28 based, at least in part, on signals from the pressure sensor 36, as generally explained in FIG. 2. An air switch stage 16′ is shown as including the air switch 84 and the electronic control circuit 28.

The CPAP machine 12 is coupled to the air switch 84, via the first hose 14 (see FIG. 1). The air switch 84 is controlled by the electronic control circuit 28, which sends a control signal to the air switch 84 to cause the air switch 84 to open, to allow incoming air from the CPAP machine 12 into the alternative CPAP mask 20″ during an inhalation portion of a user's breathing cycle and to close inhibiting incoming air from the CPAP machine 12 entering the alternative CPAP mask 20″ during an exhalation portion of a user's breathing cycle. The exhalation valve 32 together with the air switch 84 avoids the user battling an incoming rush of air from the CPAP machine 12, allowing the user to easily exhale air from the user's lungs directly into the ambient, without the exhaled air going back through the air switch 84.

Referring now to FIGS. 8A-8E, the air switch 84 is shown in various stages of operation. The air switch 84 comprises an inlet manifold 84a, a middle manifold 84c, and an outlet manifold 84b. The air switch 84 includes an air switch body 87. In FIGS. 8A-8E, the inlet manifold 84a is coupled to the CPAP machine 12 and the outlet manifold 84b is coupled to the input port 43 of the exhalation valve 32. In this embodiment, the air switch 84 does not include a diversion port that diverts air to the ambient. However in other embodiments an air switch could include a diversion port if diverting air would be the better implementation.

Referring now specifically to FIG. 8A, inlet manifold 84a has a passage 84a′ that feeds air into a middle manifold 84c through a first gasket 86a. The middle manifold 84c also houses a plunger 85 and a chamber 87a. The passage 84a′ terminates at the first gasket 86a. The plunger 85 has a bulb portion 85a and a shaft portion 85b. The bulb portion 85a has a first side 85a′ that is configured to fit tightly against a surface of the first gasket 86a, so as to present an obstruction to air flow when the air switch 84 is in a mode that blocks air flow through the air switch 84.

Disposed in the chamber 87a is a second gasket 86b on an opposing end of the bulb portion 85a. The shaft portion 85b of the plunger 85 passes through the second gasket 86b into a coil actuator platform 88. The bulb portion 85a has a second side 85a″ that is configured to fit tightly against a surface of the second gasket 86b, when the air switch 84 is in a mode that allows air flow through the air switch 84.

The end of the shaft portion 85b is affixed to the coil actuator platform 88. A soft magnetic iron pull piece 89 is affixed to a permanent magnet 90. The shaft portion 85b moves in a back and forth direction, under control of a coil, e.g., a voice coil 92 that in combination with the permanent magnet 90 and a soft magnetic iron shell 93, attracts and repels the voice coil 92. Common permanent magnet materials include hard-magnetic ferrites, Neodymium Iron Boron, and Samarium Cobalt. A fastener 94 attaches the soft magnetic iron shell 93 to the air switch body 87. The soft magnetic iron shell 93 can be any high permeability ferromagnetic material, and need not be laminated. The fastener 94 (and bonding agents) are suitable for survival in the required operating environment. By voice coil 92 is meant a mechanism including a coil that translates electrical energy into a reciprocating mechanical motion.

Passages are generally circular see FIGS. 8C and 8E (shown semi-circular in FIGS. 8A-8B-1, 8B-2, and 8D-1 and 8D-2). When the voice coil 92 conducts a current the voice coil causes a magnetic field to be generated that attracts the voice coil 92 to or from the permanent magnet 90. When the air switch is open, air at the inlet manifold 84a is inputted into the air switch 84, through the middle manifold 84c, and to the outlet manifold 84b.

In precise control applications, voice coil actuators require feedback that is provided by signals from the pressure sensors. Voice coil actuators have advantages over other kinds of actuators. For example, voice coil actuators are relatively simple in construction, e.g., do not have gears and are completely silent and do not have any backlash when direction is reversed. Also voice coil actuators are hysteresis free and provide linear motion control.

For a detailed discussion of a voice coil actuator please see Voice Coil Actuators An Applications Guide, published by BEI Kimco Magnetics Div. BEI Technologies Inc. (804-A Rancheros Dr. San Marcos CA. 92069) the entire contents of which are incorporated herein by reference.

FIG. 8B-1 shows the air switch 84 in a closed state. The closed state blocks air to freely pass through the air switch 84 by having the first side 85a′ of the plunger to fit tightly against the surface of the first gasket 86a and thus closing the passage through the air switch 84.

FIG. 8B-2 shows the air switch 84 in an open state. The open state allows input air from the CPAP machine 12 to freely pass through the air switch 84 by having the first side 85a′ of the plunger released from the surface of the first gasket 86a and thus opening the passage through the air switch 84, while also having the second side 85a″ of the plunger fitted tightly against the surface of the second gasket 86b and thus closing the passage into the coil actuator platform 88.

FIG. 8C shows the air switch 84 in an exploded view. The air switch 84 has the inlet manifold 84a attached to the middle manifold 84c that has the first gasket 86a and the second gasket 86b. The plunger 85 having the first side 85a′ that can form a tight seal with the first gasket 86a in a closed mode of operation and having the second side 85a″ that can form a tight seal with the second gasket 86b in an open mode of operation. The middle manifold supports the coil actuator platform 88, the voice coil 92, the soft magnetic pull piece 89, the permanent magnet 90 and the soft magnetic iron shell 93. The outlet manifold 84b provides an air outlet when the air switch 84 is open.

FIGS. 8D-1 and 8D-2 show perspective views of the air switch 84, with FIG. 8D-1 showing the air switch 84 closed, i.e., blocking air, and FIG. 8D-2 showing the air switch 84, open, i.e., allowing air to enter and leave the air switch 84.

FIG. 8E shows the air switch 84 with the inlet manifold 84a coupled to the middle manifold 84c and the middle manifold 84c is coupled to the outlet manifold 84b.

Reversing the current flow in the voice coil 92 causes a reversal in the interaction with the field of the permanent magnet. This allows for the voice coil 92 to move in both forward and backward or up and down (e.g., reciprocating) directions. The displacement of the voice coil 92 is proportional to the current in the voice coil 92. The proportionality of the movement of the voice coil 92 allows its use for the accurate positioning to allow for complete or partial opening of the air switch 84 to completely or partially block air from passing through the air switch 84. Although solenoids are suitable for on/off linear movement and intermittent duty, voice coils are an alternative choice to control force, speed, travel, and acceleration/deceleration for continuous performance and accurate positioning.

Elements of different implementations described herein may be combined to form other embodiments not specifically set forth above. Elements may be left out of the structures described herein without adversely affecting their operation. Furthermore, various separate elements may be combined into one or more individual elements to perform the functions described herein. Other embodiments are within the scope of the following claims. For instance, in some implementations it may be more desirable to use an alternative nasal interface or an alternative exhalation valve or an alternative micro pressure sensor, as discussed above.

One particular example of an alternative nasal interface or an alternative exhalation valve are disclosed in Ser. No. 14/632,423, filed Feb. 26, 2015, published as US-2015-0267695-A1 on Sep. 24, 2015 and entitled “Micro Pump Systems.”

One particular example of an alternative micro pressure sensor is disclosed in Ser. No. 16/163,87, filed May 2, 2018, published as US-2019-0127213-A1 on May 2, 2021 and entitled “Broad Range Micro Pressure Sensor.” Broad range as used in the publication '213 is generally at a high pressure, while the micro pressure sensor (publication '754) is at a low pressure.

In other embodiments it may be desirable to place the air switch within the CPAP mask body and the function of the positive air way pressure mask could be incorporated within a standard CPAP machine.

What is claimed is:

1. A positive air way pressure mask comprises:
a mask body having an inlet and an outlet;
a nasal interface supported on the mask body, the nasal interface having air passages, with the nasal interface coupling a user's nose to the outlet of the mask body; and
an air switch having an input port and an output port, with the input port configured to receive air from a continuous positive airway pressure machine and deliver the received air to the output port during a user inhalation, and further configured to at least partially block the air from the continuous positive airway pressure machine from passing from the input port to the output port during a user exhalation.

2. The mask of claim 1, further comprising:
an exhalation valve having an inlet port that receives air from the outlet of the air switch, the exhalation valve having an outlet port that outputs air received from a user's nostrils, and further having at least one bi-directional port disposed in an airway that couples to the air passages in the nasal interface, with an exhalation opening the outlet ports when the user exhales to minimize air resistance during the user exhaling.

3. The mask of claim 2, further comprising:
a pressure sensor that senses onset of a change in air pressure, the pressure sensor receiving air from a bi-directional port of the exhalation valve and producing in response a pressure signal; and
electronic control circuit to control operation of the air switch in response to the pressure signal received from the pressure sensor.

4. The mask of claim 1 wherein the air switch is a voice coil actuated air switch that comprises:
a switch body having a cavity;
a first gasket;
a second gasket;
a plunger having a bulb portion and a shaft portion attached to the bulb portion;
a soft magnetic, iron pull piece attached to an end of the shaft portion; and
a coil that causes the bulb portion to move into a passage in the first gasket to block air through the voice coil actuated air switch in a first mode and that causes the bulb portion to move away from the passage in the first gasket and into the second gasket to allow air to enter the voice coil actuated air switch and exit through output port of the air switch.

5. The mask of claim 4 further comprises:
a permanent magnet disposed within a region defined by the coil; and
a magnetic iron shell disposed around the coil and permanent magnet.

6. The mask of claim 4 further comprises:
a permanent magnet disposed within a region defined by the coil.

7. The mask of claim 3 wherein the electronic control circuit includes a microcontroller and the microcontroller receives signals corresponding to changes in capacitance from the pressure sensor and converts the changes in capacitance to a pressure value.

8. The mask of claim 1, further comprising:
a first hose that couples the input port of the voice coil actuated air switch to an air outlet of the positive airway pressure machine; and
a second hose that couples the output port of the voice coil actuated air switch to the air outlet of the mask body.

9. The mask of claim 3 wherein when electronic control circuit detects the end of the user's inhalation, the electronic control circuit is configured to set up for an exhalation portion of the user's breathing, initializes a delay timer, and generates after a time delay from the delay timer, a signal to control operation of a solenoid that controls operation of the air switch to divert air from the positive airway pressure machine to ambient.

10. The mask of claim 3 wherein when electronic control circuit detects the end of the user's exhalation, the electronic control circuit is configured to set up for an inhalation portion of the user's breathing, by initializing a delay timer, and generating after a time delay from the delay timer, a control signal to control operation of a solenoid controls operation of the voice coil actuated air switch to couple the air from the positive airway pressure machine to the inlet of the mask body.

11. The mask of claim 2 wherein the mask body houses the nasal interface, the air switch, and the exhalation valve.

12. The mask of claim 4 wherein the mask body houses the nasal interface, the voice coil actuated air switch, and the exhalation valve.

13. The mask of claim 3 wherein the mask body houses the nasal interface, the exhalation valve, and the pressure sensor.

14. The mask of claim 4 wherein the mask body houses the nasal interface, the voice coil actuated air switch, the exhalation valve, and the pressure sensor.

15. The mask of claim 3 wherein the mask body houses the nasal interface and the exhalation valve and wherein the pressure sensor is external to the mask body.

16. The mask of claim 4 wherein the mask body houses the nasal interface, the voice coil actuated air switch, and the exhalation valve and wherein the pressure sensor is external to the mask body.

17. A continuous positive air way pressure mask arrangement comprises:
a mask body having an inlet and an outlet;
a nasal interface supported on the mask body, the nasal interface having air passages, with the nasal interface coupling a user's nose to the outlet of the mask body; and
an air switch having an input port, an output port, and a diversion port, with the input port configurable to receive air from a continuous positive airway pressure machine and deliver the received air to the inlet of the mask body during an inhalation, and which switches the air from the positive airway pressure machine from entering the inlet port of the mask body to the diversion port diverting the received air to ambient during an exhalation.

18. The mask of claim 17, further comprising:
an exhalation valve having an inlet port that receives air from the air switch output port, the exhalation valve having an outlet port that outputs air received from a user's nostrils, and further having at least one bi-directional port disposed in an airway that couples to the air passages in the nasal interface, with exhaling opening the outlet ports when the user exhales to minimize air resistance during the user exhaling.

19. The mask of claim 17, further comprising:
a pressure sensor that senses onset of a change in air pressure, the pressure sensor receiving air from the at least one bi-directional port of the exhalation valve, and producing in response a pressure signal; and
electronic control circuit to control operation of the air switch in response to the pressure signal received from the pressure sensor.

20. The mask of claim 17 wherein the air switch comprises:
a switch body having a cavity with a generally semicircular portion;
a rotatable diversion element that forms part of a first and/or a second passage through the air switch and that is disposed in the semicircular portion and that has a channel that forms an air flow path with either the first passage or the second passage; and
a solenoid that is disposed to control rotation of the rotatable diversion element to complete or partially complete the first passage or the second passage, according to a mode of operation of the air switch.

21. A positive air way pressure mask comprises:
a mask body having an inlet and an outlet;
a nasal interface supported on the mask body, the nasal interface having air passages, with the nasal interface coupling a user's nose to the outlet of the mask body; and
a voice coil actuated air switch having an input port and an output port, with the input port configured to receive air from a continuous positive airway pressure machine and deliver the received air to the output port during a user inhalation, and further configured to at least partially block the air from the continuous positive airway pressure machine from passing from the input port to the output port during a user exhalation, the voice coil actuated air switch comprising:
a switch body having a cavity;
a first gasket;
a second gasket;
a plunger having a bulb portion and a shaft portion attached to the bulb portion;
a soft magnetic, iron pull piece attached to an end of the shaft portion; and
a coil that causes the bulb portion to move into a passage in the first gasket to block air through the voice coil actuated air switch in a first mode and that causes the bulb portion to move away from the passage in the first gasket and into the second gasket to allow air to enter the voice coil actuated air switch and exit through output port of the air switch.

22. The mask of claim 21, further comprising:
an exhalation valve having an inlet port that receives air from the outlet of the air switch, the exhalation valve having an outlet port that outputs air received from a user's nostrils, and further having at least one bi-directional port disposed in an airway that couples to the air passages in the nasal interface, with an exhalation opening the outlet ports when the user exhales to minimize air resistance during the user exhaling.

23. The mask of claim 22, further comprising:
a pressure sensor that senses onset of a change in air pressure, the pressure sensor receiving air from a bi-directional port of the exhalation valve and producing in response a pressure signal; and
electronic control circuit to control operation of the air switch in response to the pressure signal received from the pressure sensor.
